(19) United States
(12) Patent Application Publication
(10) Pub. No.: US 2025/0040891 A1
(43) Pub. Date: Feb. 6, 2025

(54) Ingestible Biosensing Capsule with Integrated Thread-Based Sensors

(71) Applicant: Trustees of Tufts College, Medford, MA (US)

(72) Inventors: Sameer Sonkusale, Lincoln, MA (US); Ruben Del Rio Ruiz, Candia, NH (US); Cihan Asci, Cambridge, MA (US)

(21) Appl. No.: 18/834,347

(22) PCT Filed: Feb. 6, 2023
(86) PCT No.: PCT/US2023/012392

§ 371 Date: Jul. 30, 2024

Related U.S. Application Data
(60) Provisional application No. 63/307,863, filed on Feb. 8, 2022.

(51) Int. Cl. A61B 5/00; A61B 5/145; A61B 5/1473; A61B 10/00; A61B 34/00

(52) U.S. Cl. CPC A61B 5/6861; A61B 5/14532; A61B 5/14539; A61B 5/14542; A61B 5/14546; A61B 5/1473; A61B 5/7285; A61B 10/0045; A61B 34/73; A61B 2010/0061; A61B 2562/125

(57) ABSTRACT

A capsule for passage through a gastrointestinal tract includes an electrochemical sensor disposed thereon and circuitry disposed within it. The circuitry uses the electrochemical sensor to obtain a measurement of a parameter within said gastrointestinal tract.

### RELATED APPLICATIONS

This application claims the benefit of the Feb. 8, 2022 priority date of U.S. Provisional Application 63/307,863, the contents of which are incorporated herein by reference.

### FIELD OF INVENTION

The invention relates to sampling the content of the gastrointestinal tract and, in particular, to sampling using an ingestible sampler.

### BACKGROUND

A human being comprises a gastrointestinal tract having a first orifice that receives food as input. As the food proceeds along the gastrointestinal tract, it undergoes various processes that result in extraction of nutrients. These nutrients are then absorbed into the human being's interior volume and used for various purposes. What remains is then ejected through a second orifice.

The gastrointestinal tract occasionally suffers from various disorders. Among these are gastric ulcers, Crohn's colitis, and inflammatory bowel disease. These diseases are often characterized by localized changes in the environment of the gastrointestinal tract. It is therefore useful, in the study and treatment of such diseases, to be able to ascertain the conditions within a limited portion of the gastrointestinal tract.

There do exist methods for observing conditions within different parts of the gastrointestinal tract. However, these tend to be invasive procedures that involve such activities as surgery, biopsy, and endoscopy. Among these procedures are those that also require considerable pre-operative cooperation from the patient, such as colonoscopy.

### SUMMARY

As one proceeds along the gastrointestinal tract, the local environmental properties change. For example, within the stomach, the environment is highly acidic. As one proceeds into the duodenum, the environment, though still acidic, begins to approach neutrality. Eventually, within the colon, the environment becomes essentially neutral.

Meanwhile, the dissolved oxygen concentration also begins to drop as one travels from the stomach towards the colon. Within the stomach, the concentration of dissolved oxygen. Within the duodenum, it will have dropped to less than half its original concentration.

As a result, it is possible to use measurements of these and other environmental variables to locate one's approximate position within the gastrointestinal tract. Knowledge of one's location makes it possible to coordinate the activity of an ingested capsule with that location, thereby making it possible to study a targeted location within the gastrointestinal tract.

In one aspect, the invention features a capsule, an electrochemical sensor disposed on the capsule, and circuitry disposed within the capsule, the circuitry being configured to use the electrochemical sensor to obtain a measurement of a parameter within the gastrointestinal tract as the capsule passes therethrough.

Embodiments include those in which the electrochemical sensor includes a conductive thread and those in which it includes a screen-printed conductor that extends along an outer surface of the capsule.

The electrochemical sensor is one that has been functionalized to make any one of a variety of measurements or combinations thereof. Among these are electrochemical sensors that have been functionalized for measurement of acidity or alkalinity, bile, glucose levels, lactate levels, dopamine levels, serotonin levels, amounts of short-chain fatty acids in the gastrointestinal tract, dissolved oxygen concentration, and concentrations of one or more neurotransmitters, all in the gastrointestinal tract. Accordingly, among the embodiments are those in which the electrochemical sensor is functionalized to sense more than one of the foregoing parameters and to do so concurrently with each other.

In some embodiments, the circuitry includes a controller and a shut-down circuit. The shut-down circuit is configured to prevent the circuitry from storing a signal provided by the electrochemical sensor.

Still other embodiments feature a sampling mechanism for drawing liquid into the capsule. In such embodiments, the controller actuates the sampling mechanism in response to information provided by the electrochemical sensor. Examples of such information include information about acidity or alkalinity, bile, glucose levels, lactate levels, dopamine levels, serotonin levels, amounts of short-chain fatty acids in the gastrointestinal tract, dissolved oxygen concentration, and concentrations of one or more neurotransmitters.

Still other embodiments include an auger and a motor that turns the auger, both of which are in the capsule. The capsule, in such embodiments, includes an opening through which the auger draws liquid. The controller is configured to start the motor automatically in response to information provided by the electrochemical sensor. Among these are embodiments in which the controller starts the motor in response to a measurement of pH, a measurement of dissolved oxygen concentration, or a combination thereof.

Also among these embodiments are those that include a reed switch. The reed switch changes state in response to an applied magnetic field so as to start the motor. This enables the controller to be overridden.

In another aspect, the invention features a method that includes providing a capsule for ingestion. This capsule has an electrochemical sensor on a surface thereof. The electrochemical sensor connects to circuitry configured to receive a signal indicative of a parameter. After the capsule has been ingested, the controller receives information from the electrochemical sensor concerning an environmental variable within a gastrointestinal tract into which the capsule has been ingested.

Among the practices are those that includes causing the controller to store information provided by the electrochemical sensor within a memory that is in the capsule and then eventually recovering the information from the memory after the capsule has been ejected from the gastrointestinal tract.

Also among the practices are those that include using the information provided by the electrochemical sensor to initiate sampling of liquid that is within the gastrointestinal tract and recovering the sample from the capsule after the capsule has been ejected from the gastrointestinal tract. Among these practices are those in which using the information includes using information indicative of pH of liquid around the capsule, using information indicative of dissolved oxygen concentration in liquid around the capsule, and using both the information indicative of dissolved oxygen concentration in liquid around the capsule and the information indicative of pH of the liquid around the capsule.

Other practices include initiating sampling of liquid that is within the gastrointestinal tract by applying a magnetic field to actuate a reed switch in the capsule and then recovering the sample from the capsule after the capsule has been ejected from the gastrointestinal tract.

In yet another aspect, the invention features using additive manufacturing to manufacture an ingestible capsule, inserting circuitry into that capsule, placing an electrochemical sensor on its outer surface, and connecting the circuitry to the electrochemical sensor.

Among these practices are those in which placing the electrochemical sensor includes placing an electrochemical sensor that is configured to measure pH, those in which placing the electrochemical sensor includes placing an electrochemical sensor that is configured to measure dissolved oxygen concentration, and those in which placing the electrochemical sensor includes placing an electrochemical sensor that is configured to measure both pH and dissolved oxygen concentration.

Also among the practices of the invention are those that further include incorporating a reed switch into the ingestible capsule to permit collection of a sample to be initiated by application of an external magnetic field.

In still another aspect, the invention features an ingestible sensor for sensing the environment of a gastrointestinal tract. Such a sensor includes a capsule, an electrochemical sensor disposed on the capsule, and circuitry disposed within the capsule. The circuitry is configured to use the electrochemical sensor to obtain a measurement of a parameter within the gastrointestinal tract.

Embodiments include those in which the electrochemical sensor comprises a conductive thread and those in which it comprises a screen-printed conductor that extends along an outer surface of the capsule.

Depending on how the electrochemical sensor has been functionalized, the measurement includes one or more of a measurement of acidity or alkalinity of the gastrointestinal tract, a measurement of one or more bile acids in the gastrointestinal tract, a measurement of glucose levels in the gastrointestinal tract, a measurement of lactate levels in the gastrointestinal tract, a measurement of dopamine levels in the gastrointestinal tract, a measurement of serotonin levels in the gastrointestinal tract, a measurement of amounts of short-chain fatty acids in the gastrointestinal tract, a measurement of oxygen in the gastrointestinal tract, and a measurement of a neurotransmitter in the gastrointestinal tract.

Further embodiments include those in which the circuitry comprises a controller and a shut-down circuit. In such embodiments, the shut-down circuit is configured to prevent the circuitry from receiving a signal provided by the electrochemical sensor.

In yet another aspect, the invention includes a method comprising providing a capsule for ingestion. The capsule includes an electrochemical sensor on a surface thereof, the electrochemical sensor being connected to circuitry configured to receive and store a signal indicative of a parameter. The method further includes, after the capsule has been ingested, causing a controller in the capsule to prevent storage of the signal.

Among the practices of the method are those that include causing the controller to enable storage of the signal and, after having done so, causing the controller to prevent storage of the signal.

Still other practices include, after having caused ingestion of the capsule, recovering the capsule.

In yet another aspect, the invention features using additive manufacturing to manufacture an ingestible capsule, inserting circuitry into the capsule, placing an electrochemical sensor on an outer surface of the capsule, and inserting circuitry into the capsule, the circuitry being connected to the electrochemical sensor. Such an additive manufacturing uses stereolithography and two-photon polymerization to promote higher resolution and finish.

In preferred practices, additive manufacturing is used for such features as a screw, a reservoir, an inlet, and an outlet. Other components, such as the thread-based flexible sensors, battery and custom flexible printed circuit boards with electronics therein are manually inserted and epoxied in place using a resin that cures when exposed to ultraviolet light.

These and other features of the invention will be apparent from the following detailed description and the accompanying figures, in which:

### BRIEF DESCRIPTION OF THE FIGURES

FIG. 1 shows an ingestible capsule having a sensor at an end thereof;

FIG. 2 is an exploded view of the capsule in FIG. 1;

FIG. 3 shows components within the middle compartment of the capsule shown in FIG. 2;

FIG. 4 shows the interconnections of the circuit components shown in FIG. 3;

FIG. 5 shows details of the shut-down circuit in FIG. 4; and

FIG. 6 shows an embodiment in which information obtained by the sensor is used to control sampling of matter from the gastrointestinal tract.

### DETAILED DESCRIPTION

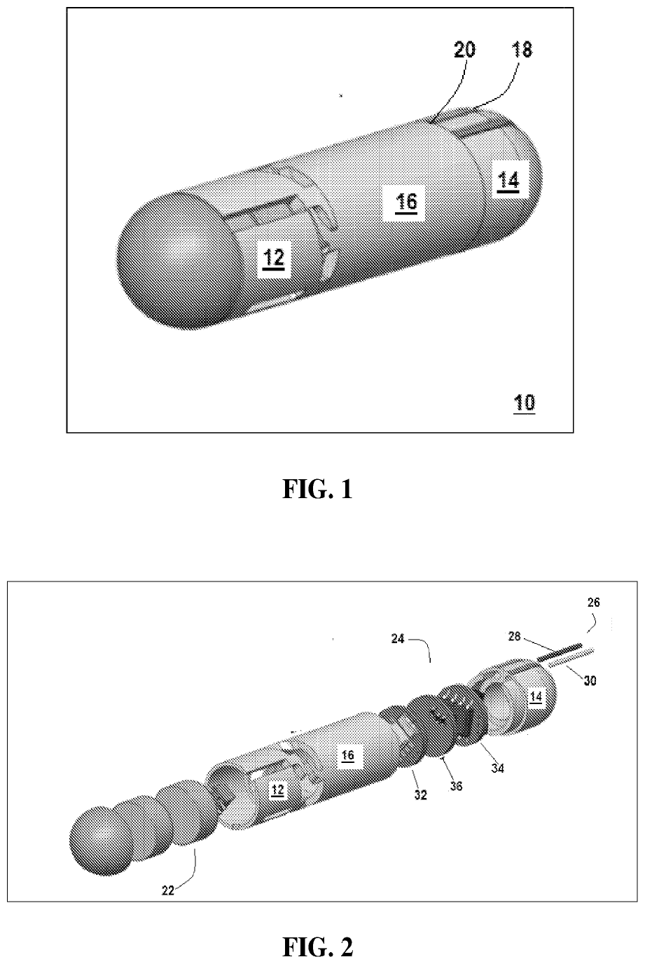

FIG. 1 shows an ingestible capsule 10 formed using an additive manufacturing process. The capsule 10 comprises first and second end-compartments 12, 14 and a middle compartment 16 therebetween. Recesses 18 on the second end-compartment 14 terminate in openings 20 that lead into the middle compartment 16.

At least a portion of the capsule 10 is fluorescent to promote its recovery after use. The capsule 10 is small enough to easily swallow. In the illustrated embodiment, the capsule 10 is thirty-three millimeters long and between eight and nine millimeters in diameter.

As shown in the exploded view of FIG. 2, the first end-compartment 12 houses a power source 22. In the illustrated embodiment, the power source 22 comprises a pair of silver oxide coin cell batteries, each of which provides 1.55 volts of potential and a total charge of 45 milliamp-hours.

On its outer surface, the second end-compartment 14 supports an electrochemical sensor 26. As shown in FIG. 2, the electrochemical sensor 26 interacts with circuitry 24 within the middle compartment 16. In the illustrated embodiment, the electrochemical sensor 26 comprises first and second threads 28, 30 nestled within the recesses 18. The first and second threads 28, 30 connect to the circuitry 24 via the openings 20.

In some embodiments, the electrochemical sensor 26 comprises conductive material that has been printed directly on the outer surface of the second end-compartment 14 during an additive manufacturing process. In such embodiments, recesses 18 are optional. The conductive material that has been printed onto the outer surface connects to the circuitry 24 through corresponding vias. In such embodiments, the second-end compartment 14 is essentially a printed-circuit board that exists on a curved manifold.

In those embodiments that are configured to sense acidity or alkalinity the first thread 28 is a reference electrode that comprises silver and the second thread 30 is a working electrode that comprises a carbon-coated linen thread that has been further coated with a conducting polymer, such as polyaniline.

Within the gut, there are many other parameters other than acidity or alkalinity that may be of interest.

For example, in some embodiments, it is useful to sense the concentration of dissolved oxygen. In addition, it is useful to be able to measure concentrations of such substances that are known to be high at selected locations since doing so provides an indication of where the capsule 10 is located. For example, an electrochemical sensor 26 that is tuned to sense bile would provide a way to recognize that the capsule 10 has reached the neighborhood at which the bile duct empties into the intestine.

The electrochemical sensor 26 is by no means limited to sensing only one parameter. After all, there is plenty of space on the second end-compartment 14 for threads 28, 30 or conductive strips that have been functionalized in different ways to sense different substances or environmental conditions within the gastrointestinal tract.

In effect, the set of all measurable properties at a location within the gastrointestinal tract defines a multi-dimensional space. Measurement of one or more coordinates of a point in that space provides information from which it is possible to make inferences concerning the location of the capsule at the time of measurement. The more coordinates one samples using the electrochemical sensor 26, the more precisely one can distinguish the location of the capsule 10. For example, if two locations have the same pH, one could not distinguish them by measuring only pH. But if one location differed from the other in dissolved oxygen concentration, then a measurement of those two coordinates in the measurement space would provide a basis for distinguishing the two locations.

The use of pH and pO₂ (i.e., partial pressure of dissolved oxygen) rather than pH alone is particularly helpful because pH varies only within a narrow range in both the small and large intestines, because it fluctuates locally, and because the spatial distribution of pH is likely to vary among individuals. The use of pH and pO₂ in concert provides enough spatial resolution to determine whether the capsule 10 is within the stomach, duodenum, jejunum, ileum, ascending colon or descending colon.

Embodiments of the electrochemical sensor 26 thus include sensors that have been configured to measure any one or more of: acidity, alkalinity, presence of bile, glucose levels, lactate levels, dopamine levels, serotonin levels, oxygen levels, and concentrations of various fatty acids, such as short-chain fatty acids.

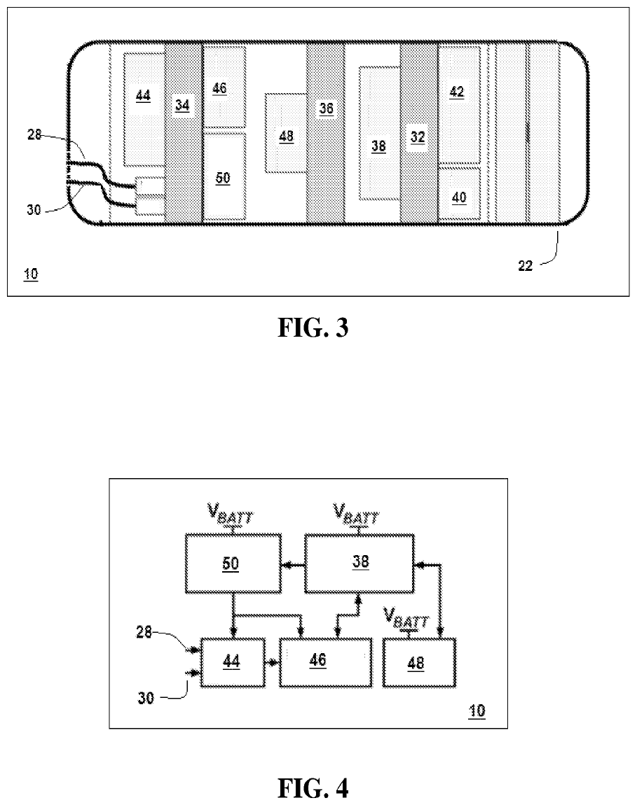

FIG. 3 shows a typical layout of the circuitry 24 within the middle compartment 16. The illustrated circuitry 24 is spread across first, second, and third printed-circuit boards 32, 34, 36. Each circuit printed-circuit boards 32, 34, 36 is a circular structure that extends across the capsule's diameter. The circuit boards 32, 34, 36 are separated along the capsule's longitudinal axis.

The first circuit board 32, which is closest to the power source 22, supports a microcontroller 38, an oscillator 40, and a programming connector 42 that permits one to program the microcontroller 38.

The second circuit board 34, which is that closest to the second end-compartment 14, interfaces with the electrochemical sensor 26 using a voltage buffer 44 that provides an analog signal to an analog-to-digital converter 46. The analog-to-digital converter 46 converts the analog signal from the electrochemical sensor 26 into data that can be stored in a memory 48, such as an EEPROM, that is on the third printed-circuit board 34. This third printed-circuit board 36 lies between the first and second printed-circuit boards 32, 34. In a preferred embodiment, the analog-to-digital converter 46 is a delta-sigma converter, and in particular, a 16-bit ΔΣ converter.

Operation of the electrochemical sensor 26 draws considerable power. In an effort to conserve the finite supply of power, it is useful to collect data only when the capsule 10 is traversing a region of interest within the tract. It is thus useful to provide a shut-down circuit 50 on the second printed-circuit board 34. This shut-down circuit 50 enables or disables operation of the analog-to-digital converter 46 and the voltage buffer 44 in response to commands from the microcontroller 38.

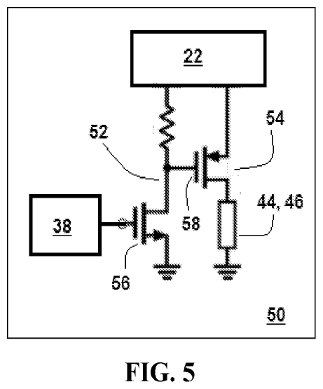

FIG. 4 is a block diagram showing the interconnection of the components discussed in connection with FIG. 3.

In response to a signal from the microcontroller 38, the shutdown circuit 50 permits the voltage buffer 44 to buffer an analog signal from the electrochemical sensor 26. It also permits the analog-to-digital converter 46 to convert that analog signal into data that it then provides the microcontroller 38. The microcontroller 38 receives this data and stores it in the memory 48. Following the capsule's ejection from the GI tract and its subsequent recovery, the data is read from the memory 48 using a suitable protocol, such as SPI or UART.

FIG. 5 shows a particular implementation of a shutdown circuit 50 featuring first and second conducting paths 52, 54, both of which connect the power source 22 to ground. The load, i.e., the voltage buffer 44 and the analog-to-digital converter 46, lies along the second conducting path 54.

First and second transistors 56, 58 that lie along the first and second conducting paths 52, 54 cause the paths 52, 54 to transition between conducting and non-conducting states. The microcontroller 38 provides a signal to the first transistor's gate. Meanwhile, the second transistor's gate connects to the first conducting path 52 at a point whose voltage depends on whether the first transistor 56 is allowing the first conducting path 52 to conduct. As a result, the microcontroller 38 also controls flow along the second conducting path 54, thereby enabling or disabling the voltage buffer 44 and the analog-to-digital converter 46.

The electrochemical sensor 26 and its associated circuitry 24 provide a basis for controlling activity by the capsule 10. In the embodiments described thus far, that activity involves gathering of measured data. However, in some embodiments, the activity involves gathering of matter, and in particular, of matter contained within the gastrointestinal tract.

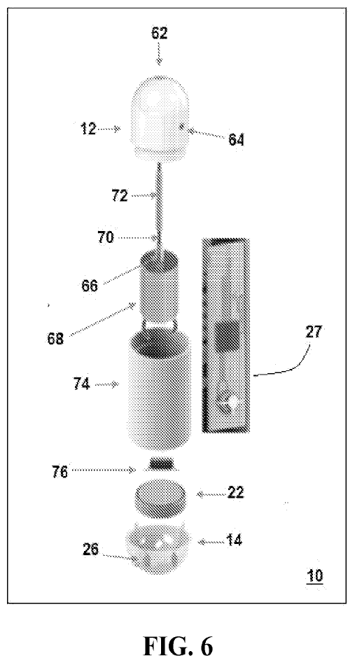

FIG. 6 shows one such embodiment of a capsule 10. In this embodiment, the electrochemical sensor 26 has been moved to surface of the first end-compartment 12. This move arises because the second end-compartment 14 is now being used for sampling. In particular, the second end-compartment 14 now has an inlet 62 for admitting liquid and an outlet 64 for allowing exit of air displaced by the incoming liquid. For those embodiments that are intended to sample from the colon, it is useful to provide an enteric coating that dissolves only upon arriving at the neutral pH environment of the colon.

A screw 66 extends along the capsule's axis between the inlet 62 and a motor 68. Such a screw 66 is typically made using an additive manufacturing process.

The screw 66 is enclosed by a bore 70 and a silicone tube 72. In operation, the motor 68 turns the screw 66. The turning screw 66 then draws liquid into the capsule through the tube 72 in a manner similar to an Archimedean screw. This liquid eventually fills a reservoir 74, displacing air through the outlet 64 as it does so.

The capsule 10 includes a switch 76 that turns the motor 68 on and off. To sample at a particular location, it is necessary to use the switch 76 to turn the motor 68 on only once the capsule 10 has reached that location.

In some embodiments, the switch 76 incorporates a reed switch that operable from outside, for example by using a magnet. This permits a clinician to override the autonomous sampling feature provided by the controller 38. Thus, a clinician could use a magnet to activate sampling or recordation of data upon confirming, using a non-invasive imaging method, (e.g., ultrasound or CT scanning) that the capsule 10 has arrived at a region-of-interest. Such an alternative activation mechanism will ensure accurate spot sampling to assess inter-individual variability in response to dietary perturbations and in patients with GI tract disturbances, which affect pH and pO₂. For example, inflammation of the gut increases pO₂. Thus, the ability to override autonomous control is particularly relevant to individuals with these types of disorders.

The motor 38 is typically a miniaturized DC motor that can turn hundreds of times per minute, thus providing rapid sampling. For example, for a typical screw 66 with four turns that traps half a microliter between adjacent blades, a readily achievable rotation rate of 1200 RPM can fill a 300-microliter reservoir in under ten seconds, independently of viscosity of the liquid being transported. This rapid sampling improves spatial resolution of the sampling process.

Preferably, the inlet 62 is sufficiently narrow to suppress movement of fluid into the capsule 10, thereby preventing contamination of the sample as the capsule continues its journey and reducing loss of the sample. It is also preferable to use a tightly sealed screw 66 inside the tube to prevent movement of fluid in either direction when the screw 66 is not turning.

The controller 38 receives information from the electrochemical sensor 26 concerning the environment surrounding the capsule 10. Based on this information, the controller 38 determines that the capsule has arrived at a desired location. The controller thus activates the switch 76, thereby initiating a sampling process. The oscillator 40 provides a basis for knowing how long sampling has taken place. After some predetermined interval, the controller 38 signals the switch 76 to turn off the motor 68, thereby halting the sampling process.

In preferred implementations, the controller 38 executes algorithms for eliminating fluctuations in the environmental parameters being measured. e.g., pH and pO₂. An example of such an algorithm is a moving average filter algorithm. The controller 38 is pre-programmed before ingestion to activate autonomously after encountering specific values of environmental parameters (e.g., pH and pO₂) over time.

Upon completing its traversal of the gastrointestinal tract, the capsule 10 is disassembled to permit the sampled liquid to be subjected to microbiota analysis. To stabilize the microbial DNA and avoid bacterial proliferation as the capsule 10 makes its way to the end of the gastrointestinal tract, it is useful to provide a small volume of chaotropic salt in the reservoir 74.

Having described the invention and a preferred embodiment thereof, what is claimed as new and secured by Letters Patent is:

1. An apparatus for passage through a gastrointestinal tract, said apparatus comprising a capsule, an electrochemical sensor disposed on said capsule, and circuitry disposed within said capsule, said circuitry being configured to use said electrochemical sensor to obtain a measurement of a parameter within said gastrointestinal tract.

2. The apparatus of claim 1, wherein said electrochemical sensor comprises a conductive thread.

3. The apparatus of claim 1, wherein said electrochemical sensor comprises a screen-printed conductor that extends along an outer surface of said capsule.

4. The apparatus of claim 1, wherein said electrochemical sensor is functionalized for measurement of acidity or alkalinity of said gastrointestinal tract.

5. (canceled)

6. The apparatus of claim 1, wherein said measurement is a measurement of glucose levels in said gastrointestinal tract.

7. (canceled)

8. The apparatus of claim 1, wherein said measurement is a measurement of dopamine levels in said gastrointestinal tract.

9. The apparatus of claim 1, wherein said electrochemical sensor is functionalized for measurement of serotonin levels in said gastrointestinal tract.

10. The apparatus of claim 1, wherein said electrochemical sensor is functionalized for measurement of amounts of short-chain fatty acids in said gastrointestinal tract.

11. The apparatus of claim 1, wherein said electrochemical sensor is functionalized for measurement of dissolved oxygen concentration in said gastrointestinal tract.

12. The apparatus of claim 1, wherein said wherein said electrochemical sensor is functionalized for measurement of a neurotransmitter in said gastrointestinal tract.

13. The apparatus of claim 1, wherein said circuitry comprises a controller and a shut-down circuit, said shut-down circuit being configured to prevent said circuitry from receiving a signal provided by said electrochemical sensor.

14. The apparatus of claim 1, wherein said electrochemical sensor is functionalized to sense plural parameters.

15. The apparatus of claim 1, further comprising a sampling mechanism for drawing liquid into said capsule, wherein said controller is configured to actuate said sampling mechanism in response to information provided by said electrochemical sensor.

16. The apparatus of claim 1, further comprising an auger and a motor that turns said auger, wherein said capsule comprises an opening through which said auger draws liquid, wherein said controller is configured to start said motor automatically in response to information provided by said electrochemical sensor.

17. The apparatus of claim 1, wherein said controller is configured to start sampling automatically in response to a measurement of pH provided by said electrochemical sensor.

18. The apparatus of claim 1, wherein said controller is configured to start sampling automatically in response to a measurement of dissolved oxygen concentration provided by said electrochemical sensor.

19. The apparatus of claim 1, wherein said controller is configured to start sampling automatically in response to measurements of both dissolved oxygen concentration and pH, said measurements having been provided by said electrochemical sensor.

20. The apparatus of claim 1, further comprising a reed switch that is configured to change state in response to an applied magnetic field so as to start sampling, thereby enabling said controller to be overridden.

21. A method comprising providing a capsule for ingestion, said capsule comprising an electrochemical sensor on a surface thereof, said electrochemical sensor being connected to circuitry configured to receive a signal indicative of a parameter and, after said capsule has been ingested, receiving information from said electrochemical sensor concerning an environmental variable within a gastrointestinal tract into which said capsule has been ingested.

22-27. (canceled)

28. A method comprising using additive manufacturing to manufacture an ingestible capsule, inserting circuitry into said capsule, placing an electrochemical sensor on an outer surface of said capsule, and connecting said circuitry to said electrochemical sensor.

29-32. (canceled)
